(19) United States
(12) Patent Application Publication
(10) Pub. No.: US 2016/0051777 A1
(43) Pub. Date: Feb. 25, 2016

(54) METERING ELEMENT FOR AN INHALATION DEVICE AND ASSEMBLY FOR AN INHALATION DEVICE CONTAINING SAME

(71) Applicant: SANOFI SA, Vernier (CH)

(72) Inventor: Stefan Mayer, Frankfurt am Main (DE)

(21) Appl. No.: 14/779,123

(22) PCT Filed: Apr. 2, 2014
(86) PCT No.: PCT/EP2014/056621

§ 371 Date: Sep. 22, 2015

(30) Foreign Application Priority Data
Apr. 3, 2013 (EP) 13162150.0

(51) Int. Cl. A61M 15/00

(52) U.S. Cl. CPC A61M 15/0066; A61M 15/002; A61M 2202/064; A61M 2205/3334

(57) ABSTRACT

A metering element for an inhalation device is provided. The metering element comprises a plurality of openings being configured to receive a substance, wherein at least one of the openings has a different size than at least one other opening. Furthermore, an assembly for an inhalation device is provided. The assembly comprises a metering element with a plurality of openings and a powder channel which comprises an opening, wherein an arrangement of the openings is adapted to the shape of the powder channel such that each opening extends into the opening of the powder channel for a different amount.

### CROSS REFERENCE TO RELATED APPLICATIONS

The present application is a U.S. National Phase Application pursuant to 35 U.S.C. §371 of International Application No. PCT/EP2014/056621 filed Apr. 2, 2014, which claims priority to European Patent Application No. 13162150.0 filed Apr. 3, 2013. The entire disclosure contents of these applications are herewith incorporated by reference into the present application.

### TECHNICAL FIELD

The present disclosure relates to a metering element for an inhalation device and an inhalation device comprising a metering element.

### BACKGROUND

A metering element for an inhalation device is known from document WO 2009/065707 A1. This application relates to a metering device which can be activated by the suction airflow of a user for inhaling a powdery substance which is arranged in a supply chamber.

### SUMMARY

It is an object of the present invention to provide a metering element for an inhalation device having improved properties.

According to one aspect of the disclosure, a metering element for an inhalation device is provided. The metering element comprises a plurality of openings being configured to receive a substance, wherein at least one of the openings has a different size than at least one other opening. The inhalation device may be used for the inhalation of a substance. The substance may be stored in storage chamber of the device. The openings may be configured to receive a predetermined amount of the substance. Thereby, an amount of the substance which is required for one inhalation may be measured.

The advantage of a metering element comprising differently sized openings is that the substance may flow out of the openings during an inhalation with a different flow rate. The flow rate correlates to the particles of the substance which flow out of an opening in a particular time. Furthermore, the substance may have a different adherence in the different sized openings. Thereby, the substance may be delivered to a powder channel of the inhalation device from the different openings time delayed. Thereby, a distribution of the substance in the powder channel may be improved. Thereby, the substance which is inhaled by a user may comprise a sufficient fine particle fraction. Thereby the fine particle dose which is inhaled during one inhalation is increased. Thereby, the effect of the inhalation may be improved.

According to one embodiment each opening may have a size different from the other openings. According to an alternative embodiment, each opening may have the same size as the other openings. The metering element may comprise at least three openings. For example the metering element comprises three openings.

According to one embodiment, at least one of the openings may have the form of an ellipse. Preferably, all openings have the form of an ellipse. In an alternative embodiment, at least one of the openings may have the form of a circle. The form of the openings may have an influence of the adherence of the substance in the openings.

According to one embodiment, the openings are arranged in a pattern of a circle. Alternatively, the openings may be arranged in a pattern of a circle, a rectangle or a rhomb. In particular, the openings may be arranged at an end of the powder channel of the inhalation device and partially extend into the powder channel when the device is ready for an inhalation. At the end of the powder channel, the powder channel may comprise an opening such that the substance may flow into the powder channel from the openings of the metering element. In particular, the metering element may abut or be arranged next to an end of the powder channel.

According to one embodiment, the distance of an opening to its adjacent openings is the same. In particular, each opening has the same distance to its adjacent openings. The distance of an opening to its adjacent openings may be small, for example 1 mm. In particular, the distance of an opening to its adjacent openings may be as small as possible due to the producibility of the metering element, in particular the openings.

According to one embodiment, the metering element is configured to be moved in a moving direction. For this purpose, the metering element may comprise a knob. The knob may be clasped by another element of the device. By means of the knob, a movement may be transferred to the metering element from another element of the device. The metering element may be configured as a rod. Preferably, the metering element is configured as a flat bar. The metering element may have a rectangular cross-section. Alternatively, the metering element may have a circular cross-section.

The metering element may be configured to move axially in a moving direction. The moving direction may be a direction along the longitudinal axis of the metering element. In an alternative embodiment, the moving direction may be a direction tilted with respect to the longitudinal axis, in particular perpendicular to the longitudinal axis of the metering element. Furthermore, the metering element may rotate with respect to its longitudinal axis. For delivering a dose of substance, the metering element may be moved along the longitudinal axis towards a dispensing end of the device. After use, the metering element may be moved away from the dispensing end of the device.

Preferably, the metering element is configured to measure a sub-quantity of a substance from a total quantity of substance. In particular, the metering element may receive a sub-quantity of substance via the opening. The substance may be a powder. The substance may be a medicament.

In a preferred embodiment, the metering element comprises a plurality of metering chambers. The metering chambers may be cavities provided in the metering element. The cavities may be conical. Thereby, the substance may easily run into the metering chamber. Preferably, the openings lead into the metering chambers. The metering chambers may be configured to admeasure a sub-quantity of the substance.

Preferably, the openings are arranged eccentrically on the metering element with respect to the longitudinal axis. Thereby, the metering chambers may act as shovels gathering substance when the metering element is rotated. On rotation and axial movement of the metering element the openings, respectively the metering chambers, move through an accumulation of substance helicoidally. In particular, the openings may be moved along a screw curve when the metering element performs a combined axial and rotational movement. Thereby, an adequate filling of the metering chambers with substance may be achieved on rotation and axial movement of the metering element.

According to one aspect of the invention, an assembly for an inhalation device is provided. The assembly comprises a metering element which comprises a plurality of openings. Furthermore, the assembly comprises a powder channel. Via the powder channel, substance may be dispensed to a user by means of an airflow. The airflow may be generated when a user inhales. The arrangement of the openings is adapted to the shape of the powder channel such that each opening extends into the opening of the powder channel for a different amount. In particular, the openings may be arranged around the powder channel. The metering element may be configured as previously described.

During an inhalation, the sub-quantity of substance may flow through the powder channel. Preferably, the flow profile of the substance in the powder channel is influenced by the shape and the arrangement of the openings. The flow profile may describe the velocity and the path of the particles of the substance. Furthermore, the flow profile of the substance in the powder channel may be influenced by the shape of the metering chamber. Preferably, the substance comprises a fine particle fraction. Preferably, the flow profile has an influence on the composition of the fine particle fraction of the substance.

The advantage of an assembly for an inhalation device wherein each opening extends into the powder channel for a different amount is that a distribution of a substance in the powder channel may be improved. In particular, the substance may be dispensed into the powder channel with a different flow rate from each opening. Thereby, a high amount of the substance which is dispensed into the powder channel during one inhalation may strive along an interior wall of the powder channel. Thereby, a deaggregation of the substance may be achieved. Thereby, the substance which is inhaled by a user may comprise a sufficient fine particle fraction. In particular, the separation of a micronized portion of the substance from a carrier material is achieved. Thereby, the medical effect of an inhalation may be improved.

According to one embodiment, the assembly may comprise a storage chamber. The storage chamber is configured to contain a quantity of substance. In particular, the storage chamber may be configured to contain an amount of substance which corresponds to a plurality of sub-quantities of substance.

The metering element is configured to transport a sub-quantity of substance out of the storage chamber. In particular, the metering element may be configured to transport a sub-quantity of substance out of the storage chamber via the metering chamber. In particular the metering element may be configured to transport a sub-quantity of substance into the powder channel.

Preferably, the metering element is configured to rotate and axially move with respect to the storage chamber. The metering element may axially move from a first position inside the storage chamber to a second position outside the storage chamber. The first position may be a proximal position of the metering element furthest away from the dispensing end of the device. In the first position the metering element is at least partially positioned in the storage chamber. In particular, at least the metering chamber is fully dipped in the storage chamber. The second position may be a distal position of the metering element nearest to the dispensing end of the device. In the second position, the metering element is positioned outside of the storage chamber. In particular, the metering chamber is positioned outside of the storage chamber.

Preferably, the metering element gathers a sub-quantity of substance from the storage chamber on rotation, in particular on rotation and axial movement. The metering element may gather a sub-quantity of substance when it is in the storage chamber. Preferably, the metering element transports a sub-quantity of substance out of the storage chamber on axial movement.

According to one embodiment, the openings may be partially covered when the metering element is in a position furthest away from the storage chamber, in particular in its second position. The openings may be partially covered by a wall of the powder channel or by another component of the device. Thereby, an intake pressure which has to be built up by a suction air flow of a user needs to be sufficient to draw the substance through the opening. In particular, the more an opening is covered, the higher the intake pressure needs to be in order to draw the substance through the opening.

According to one embodiment, each opening comprises a size different from the other openings. The area of an opening which is covered may be dependent on the size of the opening. In particular, the larger an opening, the larger is the area of the opening which is covered. In particular, the area which is covered may be different for each opening. According to a further embodiment, each opening comprises the same size. In such an embodiment, a different area of each opening is covered independently from the size of the opening.

According to one embodiment, the larger an opening is, the more it extends into the powder channel. In particular for a metering element with different sized openings, the largest opening extends into the powder channel most. During an inhalation, the substance may be drawn from the opening which extends most into the powder channel first. The substance may be drawn from the opening which extends least into the powder channel at last. Thereby, a disposal of the substance from the different openings into the powder channel may happen time-delayed. According to one embodiment, the disposal of the substance from the different openings may happen one after another and not simultaneously. Thereby, the distribution of the substance in the powder channel may be improved.

According to one embodiment, the disposal of the substance from the different openings into the powder channel may happen with a different flow rate. In particular, the less an opening extends into the powder channel, the smaller is the flow rate with which the substance flows into the powder channel.

According to a further aspect of the disclosure, an inhalation device comprising the above disclosed assembly is provided.

The term “substance”, as used herein may mean a pharmaceutical formulation containing at least one pharmaceutically active compound, for example for the treatment of obstructive airway or lung diseases such as asthma or chronic obstructive pulmonary disease (COPD), local respiratory tract oedema, inflammation, viral, bacterial, mycotic or other infection, allergies, diabetes mellitus.

The active pharmaceutical compound is preferably selected from the group consisting of active pharmaceutical compounds suitable for inhalation, preferably antiallergenic, antihistamine, anti-inflammatory, antitussive agents, bronchodilators, anticholinergic drugs, and combinations thereof.

The active pharmaceutical compound may for example be chosen from:

an insulin such as human insulin, e.g. a recombinant human insulin, or a human insulin analogue or derivative, a glucagon-like peptide (GLP-1) or an analogue or derivative thereof, or exendin-3 or exendin-4 or an analogue or derivative of exendin-3 or exendin-4;

an adrenergic agent such as a short acting β2-agonists (e.g. Salbutamol, Albuterol, Levosalbutamol, Fenoterol, Terbutaline, Pirbuterol, Procaterol, Bitolterol, Rimiterol, Carbuterol, Tulobuterol, Reproterol), a long acting β2-agonist (LABA, e.g. Arformoterol, Bambuterol, Clenbuterol, Formoterol, Salmeterol), an ultra LABA (e.g. Indacaterol) or another adrenergic agent (e.g. Epinephrine, Hexoprenaline, Isoprenaline (Isoproterenol), Orciprenaline (Metaproterenol));

a glucocorticoid (e.g. Beclometasone, Budesonide, Ciclesonide, Fluticasone, Mometasone, Flunisolide, Betamethasone, Triamcinolone);

an anticholinergic agent or muscarinic antagonist (e.g. Ipratropium bromide, Oxitropium bromide, Tiotropium bromide);

a mast cell stabilizer (e.g. Cromoglicate, Nedocromil);

a xanthine derivative (e.g. Doxofylline, Enprofylline, Theobromine, Theophylline, Aminophylline, Choline theophyllinate);

an eicosanoid inhibitor, such as a leukotriene antagonist (e.g. Montelukast, Pranlukast, Zafirlukast), a lipoxygenase inhibitor (e.g. Zileuton) or a thromboxane receptor antagonist (e.g. Ramatroban, Seratrodast);

a phosphodiesterase type-4 inhibitor (e.g. Roflumilast); an antihistamine (e.g. Loratadine, Desloratadine, Cetirizen, Levocetirizine, Fexofenadine);

an allergen immunotherapy (e.g. Omalizumab);

a mucolytic (e.g. Carbocisteine, Erdosteine, Mecysteine);

an antibiotic or antimycotic;

or a combination of any two, three or more of the above-mentioned compound classes or compounds (e.g. Budesonide/Formoterol, Fluticasone/Salmeterol, Ipratropium bromide/Salbutamol, Mometasone/Formoterol);

or a pharmaceutically acceptable salt or solvate or esters of any of the above named compounds.

Pharmaceutically acceptable salts are for example acid addition salts and basic salts. Acid addition salts are e.g. a chloride, bromide, iodide, nitrate, carbonate, sulfate, methylsulfate, phosphate, acetate, benzoate, benzenesulfonate, fumarate, malonate, tartrate, succinate, citrate, lactate, gluconate, glutamate, edetate, mesylate, pamoate, pantothenate or a hydroxy-naphthoate salt. Basic salts are for example salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology. Pharmaceutically acceptable ester may for example be acetates, propionates, phosphates, succinates or etabonates.

Pharmaceutically acceptable solvates are for example hydrates.

### BRIEF DESCRIPTION OF THE FIGURES

Further features and refinements become apparent from the following description of the exemplary embodiments in connection with the figures.

FIG. 1 schematically shows a sectional view of an inhalation device,

FIG. 2 shows a metering element,

FIG. 3 shows a section of the metering element of FIG. 2.

### DETAILED DESCRIPTION

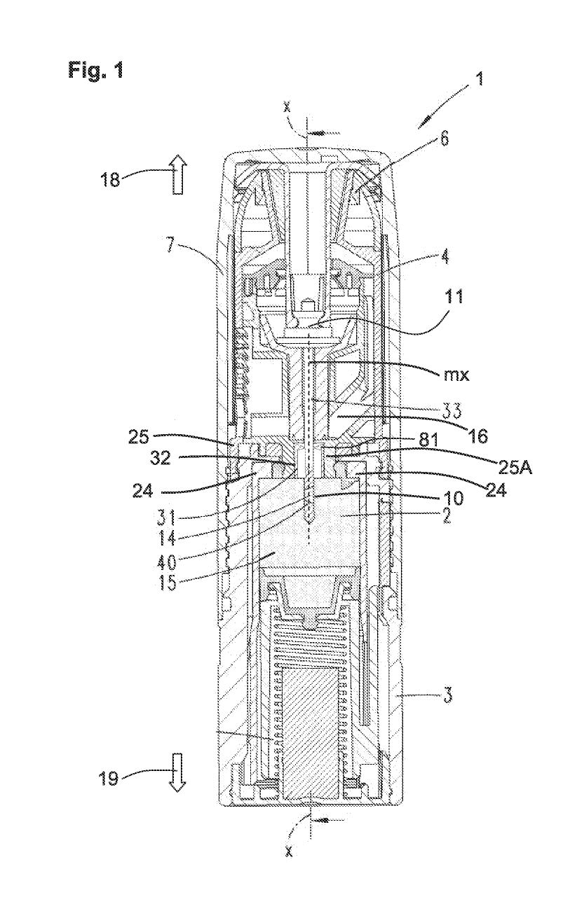

FIG. 1 shows a sectional view of an inhalation device 1. The inhalation device 1 is configured to be activated by a suction airflow generated by a user. The inhalation device 1 comprises a housing 3. Furthermore, the device 1 comprises an outer cylinder 4. The outer cylinder 4 is secured against axial movement with respect to the housing 3. The outer cylinder 4 is rotatable with respect to the housing 3.

Furthermore, the inhalation device 1 comprises a mouthpiece 6. Via the mouthpiece 6, air is sucked into the inhalation device 1. The inhalation device 1 further comprises a cap 7. The cap 7 may be configured as a screw cap. The cap 7 is used for covering the mouthpiece 6. The cap 7 may be rotatable about a main longitudinal axis x of the inhalation device 1 in a first direction with respect to the housing for screwing the cap 7 onto the device 1 and in a second direction with respect to the housing 3 for unscrewing the cap 7 from the device 1. The outer cylinder 4 is rotationally connected to the cap 7. In particular, the outer cylinder 4 follows a rotation of the cap 7 with respect to the housing 3. For a detailed description of the components of the inhalation device 1 and their mechanical cooperation it is referred to document WO 2009/065707 A1, the entire content of which is explicitly incorporated by reference into the present description, in particular as far as the operation of the device 1 is concerned.

The device 1 further comprises a storage chamber 15. The storage chamber 15 holds at least one dose of a substance 2. In particular, the storage chamber 15 may hold a plurality of doses of a substance 2. The substance 2 may comprise a drug. The substance 2 may comprise a powder.

The storage chamber 15 is terminated by a chamber sealing 24. In particular, the side of the storage chamber 15 which is faced towards the mouthpiece is terminated by the chamber sealing 24. The device 1 further comprises a rotary part 25. The rotary part 25 is connected in a rotationally fixed manner to the outer cylinder 4. Accordingly, the rotary part 25 follows a rotation of the outer cylinder and, hence, of the cap 7 about the main longitudinal axis x with respect to the storage chamber 15.

The chamber ceiling 24 comprises a central through opening. A cylindrical portion 25A of the rotary part 25 passes through the central through opening of the chamber ceiling 24.

The inhalation device 1 further comprises a metering element 33. The metering element 33 may comprise a metering rod. The metering element 33 may have a circular or a non-circular cross-section. For example, the metering element 33 may have a rectangular cross-section.

The metering element 33 comprises a longitudinal axis mx. The longitudinal axis mx of the metering element 33 is parallel to the main longitudinal axis x of the device 1. In particular, the longitudinal axis mx coincides with the main longitudinal axis x of the device 1. The metering element 33 is axially and rotationally movable with respect to the storage chamber 15. When the cap 7 is demounted from the device 1, i.e. during an operation of the device 1, the metering element 33 is moved in a distal direction 18. The distal direction 18 is a direction towards a dispensing end of the device. When the cap 7 is remounted onto the device 1, i.e. after an operation was completed, the metering element 33 is moved in a proximal direction 19. The proximal direction 19 is a direction away from the dispensing end of the device. The metering element 33 is rotationally connected to the rotary part 25 by mechanical cooperation with the rotary part 25. Accordingly, the metering element 33 follows rotational movement of the cap 7 and, hence, of the rotary part 25 about the main longitudinal axis x when the cap 7 is mounted onto the device 1 or demounted from the device 1.

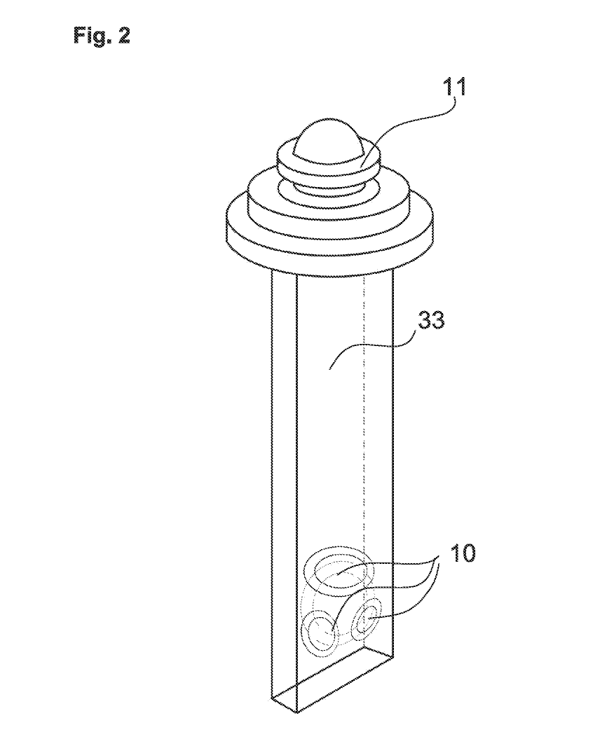

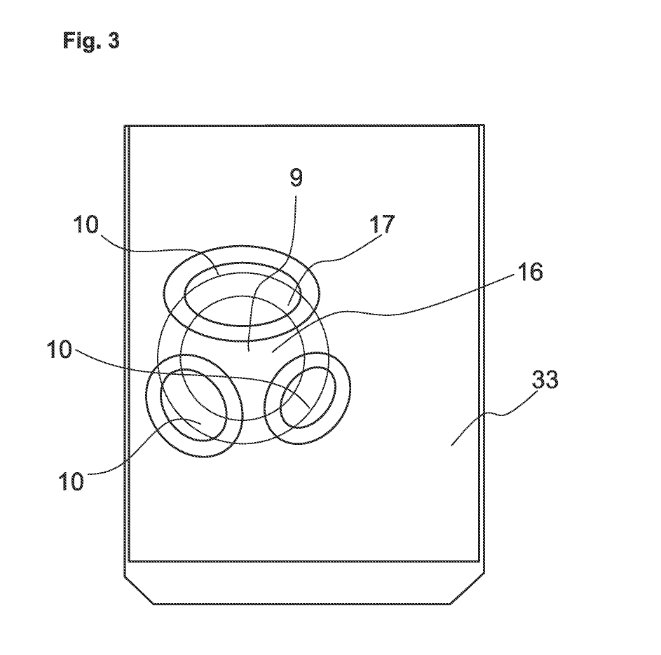

The metering element 33 comprises at least one metering chamber 40. The metering chamber 40 is located near a proximal end of the metering element 33. The proximal end of the metering element 33 is the end, which his located in the storage chamber 15 when the cap 7 is mounted on the device. The metering element 33 is configured for moving the metering chamber 40 from a first position, wherein the metering chamber 40 is located inside the storage chamber 15, to a second position, wherein the metering 40 is located outside of the storage chamber 15. The metering chamber 40 is configured for measuring and accommodating a sub-quantity 14 of the substance 2 which is to be dispensed during an inhalation action performed by a user. In particular, a sub-quantity 14 of the substance 2 may be transported from the storage chamber 15 to a powder channel 16 via the metering chamber 40. In order to collect a sub-quantity 14 of the substance 2, each metering chamber 40 comprises an opening 10. As can be seen in FIGS. 2 and 3, the openings 10 are arranged eccentric with respect to the axis mx on the metering element 33, in order to achieve an adequate filling of the metering chambers 40 with substance 2. In particular, the metering chambers 40 helicoidally move through the storage chamber 15, thereby gathering a sub-quantity 14 of substance 2.

For a detailed description of the operation of the metering element 33, it is referred to document WO 2009/065707 A1.

FIG. 2 shows a metering element 33 which is configured for use in an inhalation device 1 as described with reference to FIG. 1. The metering element 33 comprises three openings 10. The openings 10 have a different size.

Furthermore, the metering element 33 comprises a knob 11. The knob 11 serves as a holding element. In particular, the metering element 33 is mounted in the device by means of the knob 11. Furthermore, the metering element 33 may be moved by means of the knob 11. The knob 11 is clasped by another element of the device, as shown in FIG. 1.

FIG. 3 shows a detailed view of the part of the metering element 33 comprising the openings 10. Furthermore, the position of the powder channel 16 relative to the metering element 33 is indicated. In particular, the profile of a wall 17 of the powder channel 16 is shown. In FIG. 3, the metering element 33 is shown in a relative position with respect to the powder channel 16 when the metering element 33 has been moved out of the storage chamber 15. In particular, a sub-quantity of substance 2 may be delivered to the powder channel 16 when the metering element 33 is in the position shown in FIG. 3.

The openings 10 of the metering element each have the form of an ellipse. The openings 10 are arranged along the circular opening 9 of the powder channel 16. In particular, the openings 10 extend into the powder channel 16. Furthermore, the openings 10 are partially covered, for example by the wall 17 of the powder channel 16 or by another component of the device.

Each opening 10 of the metering element extends into the opening 9 of the powder channel 16 for a different amount. This amount may depend on the size of each respective opening 10. In particular, the larger the size of an opening 10, the more the opening 10 extends into the opening 9 of the powder channel 16.

In each metering chamber 40, a different pressure ratio is developed during an inhalation. Thereby, the substance 2 flows into the powder channel 16 against a different flow resistance when a user inhales. In particular, the opening 10 which extends least into the opening 9 of the powder channel 16 generates the highest flow resistance. The opening 10 which extends least into the opening 9 of the powder channel 16 is the smallest opening 10. Furthermore, the smaller the metering chamber 40, the more the substance 2 adheres to an interior wall of the metering chamber 40.

The different flow resistance effects that the substance 2 is delivered to the powder channel 16 from each opening 10 time-delayed. When a user inhales, the substance 2 from the opening 10 which extends into the opening 9 of the powder channel 16 for the greatest amount, i. e. the largest opening 10, flows into the powder channel 16 first, and the substance 2 from the opening 10 which extends into the opening 9 of the powder channel 16 for the smallest amount, i. e. the smallest opening 10, flows into the powder channel 16 at last. Thereby, a good distribution of the substance 2 in the powder channel 16 is achieved. Since the substance 2 is delivered from the openings 10 into the powder channel 16 consecutively instead of simultaneously, a large amount of the substance 2 may strive along an interior wall of the powder channel 16. Thereby, the substance 2 may be deaggregated. Thereby, the substance 2 which is inhaled by a user may comprise a sufficient fine particle fraction. In particular, the separation of the micronized portion of the substance from a carrier material is achieved.

1-15. (canceled)

16. A metering element for an inhalation device, comprising a plurality of openings being configured to receive a substance, wherein at least one of the openings has a different size than at least one other opening, wherein the openings are configured such that the substance flows out of the openings during an inhalation with a different flow rate.

17. The metering element according to claim 16, wherein each opening has a different size than the other openings.

18. The metering element according to claim 16, wherein at least one of the openings comprises the form of an ellipse.

19. The metering element according to claim 16, comprising three openings.

20. The metering element according to claim 16, wherein the openings are arranged in a pattern of a circle.

21. The metering element according to claim 16, wherein the distance of an opening to its adjacent openings is the same.

22. The metering element according to claim 16 which is configured as a flat bar.

23. An assembly for an inhalation device, comprising the metering element according to claim 1 and a powder channel comprising an opening, wherein an arrangement of the openings is adapted to the shape of the powder channel such that each opening extends into the opening of the powder channel for a different amount.

24. The assembly according to claim 23, comprising a storage chamber containing a total quantity of a substance, wherein the metering element is configured to transport a sub-quantity of substance out of the storage chamber.

25. The assembly according to claim 23, wherein the metering element is configured to transport a sub-quantity of the substance into the powder channel.

26. The assembly according to claim 24, wherein the openings are partially covered when the metering element is in a position furthest away from the storage chamber.

27. The assembly according to claim 23, wherein each opening comprises a size different from the other openings.

28. The assembly according to claim 27, wherein the larger an opening, the more it extends into the powder channel.

29. The assembly according to claim 23, wherein a disposal of the substance from the different openings into the powder channel happens time-delayed.

30. The assembly according to claim 23, wherein the disposal of the substance from the different openings into the powder channel happens with a different flow rate
